(19) United States
(12) Patent Application Publication
(10) Pub. No.: US 2024/0351985 A1
(43) Pub. Date: Oct. 24, 2024

(54) NOVEL COMPOUND AND USE OF SAID COMPOUND

(71) Applicants: APHEA.BIO NV, Zwijnaarde (BE); Fundación Centro De Excelencia En Investigación De Medicamentos Innovadores En Andalucía, Medina, Granada (ES)

(72) Inventors: Ignacio PÉREZ-VICTORIA MORENO DE BARREDA, Granada (ES); José Rubén TORMO BELTRÁN, Madrid (ES); Steven VANDENABEELE, Zwijnaarde (BE); Thomas SIMON, Zwijnaarde (BE); Isabel VERCAUTEREN, Zwijnaarde (BE); Victor Manuel GONZÁLEZ MENÉNDEZ, Armilla, Granada (ES)

(21) Appl. No.: 18/684,596

(22) PCT Filed: Aug. 18, 2022
(86) PCT No.: PCT/EP2022/073042

§ 371 Date: Feb. 16, 2024

(30) Foreign Application Priority Data
Aug. 18, 2021 (EP) 21191906.3

(51) Int. Cl. C07D 211/42; A01N 37/36; A01N 43/40; A01P 3/00; C07C 233/05; C12N 1/20; C12R 1/465

(52) U.S. Cl. CPC C07D 211/42; A01N 37/36; A01N 43/40; A01P 3/00; C07C 233/05; C12N 1/205; C12R 2001/465

(57) ABSTRACT

The current invention is directed to a novel compound and compositions having said compound, wherein said compound is a compound according to formula I or formula II, or an acceptable salt thereof. The invention also relates to specific uses of said compound, particularly as antimicrobial agent.

### FIELD OF THE INVENTION

The present invention relates to a novel compound and uses thereof, wherein said compound is produced by bacterial strains such as Streptomyces sp.

### BACKGROUND

Plant diseases, caused by fungi and bacteria, represent a major threat to economically important agricultural crops, like cereals, fruits and vegetables. The yield of plants, for example, wheat, maize, rice, soybean, grape, apple and tomato, are adversely impacted by fungal and bacterial diseases. Disease management by application of fungicides is mandatory for a profitable yield but repeated usage of a single fungicide often leads to the development of resistance of the pathogen.

Bacterial antibiotic resistance is an even stronger issue going beyond the agricultural and food industries, to the medical and veterinary sectors, and others. The potential for transfer of antibiotic resistance, or of potentially lethal antibiotic-resistant bacteria from a food animal to a human consumer is of particular concern. Current methods for controlling development and spread of antibiotic-resistant bacteria include changes in antibiotic usage and patterns of usage of different antibiotics, increased governmental surveillance and regulation, and continued development of new and improved antibiotics. However, the ability of most bacteria to adapt to antibiotic usage and to acquire resistance to existing and new antibiotics often overcomes such conventional measures, and requires the continued development of alternative means for control of antibiotic resistance in bacteria.

Therefore, there is a continuing need to provide new compositions having antimicrobial activity for controlling fungal and bacterial pathogens, thereby indirectly increasing the yield of plants. These compounds should preferably be safe and stable and derived from nature.

The current invention aims to provide a solution for at least one of the problems mentioned above, by proposing a compound efficient in combatting fungal and bacterial diseases in plant, and that is derivable from Streptomyces sp.

### SUMMARY OF THE INVENTION

The present invention and embodiments thereof serve to provide a solution to one or more of above-mentioned needs. To this end, the present invention relates to a novel compound according to claim 1.

This invention further relates to a composition that comprises the novel compound according to claim 4 and a microorganism producing the novel compound according to claim 8.

The invention also relates to specific uses as described in any of claims 10 to 13. Said compound has a broad action against fungal and bacterial pathogens, and can be used for treatment of infections in plants and humans, particularly for agriculture applications.

The invention also relates to a method according to any of claims 14 to 15.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The term “pathogen” as used herein relates to a causative agent of disease. Such causative agent can be, amongst others, a microorganism such as a virus, a bacterium or a fungi. The term “pest” or “plant pest” refers to a destructive insect or other animal that attacks and (partially) destroys plants. Most important plant pests are insects, mites, nematodes and gastropod molluscs. Plant pests may also transmit fungal, bacterial or viral infections. As used herein, pathogen and pest may be used interchangeably and may refer to both pathogens and pests as defined before.

The term “soil sourced” relates to an organism that originally originates from the soil, preferably from the root environment of plants and were sourced from the latter. Soil sourced organisms such as bacteria may then be further cloned or propagated in an environment outside their initial environment, such as a laboratory environment.

As used herein, the term “microorganism” or “microbe” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms) or more than one cell (e.g., multi-cellular microorganism).

As used herein, the term “bacterium”, “bacteria”, or “bacterial” refers in general to any prokaryotic organism, and may reference an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

The term “purified” is intended to specifically reference an organism, cell, tissue, polynucleotide, or polypeptide that is removed from its original source. The term “purified” does not necessarily reflect the extent to which the microbe has been purified.

As used herein, a “purified bacteria” or “purified bacterial strain” is a bacterial strain that has been removed from its natural milieu. The term “purified bacterial strain” refers to substantially no other strains than the desired strain, and is therefore substantially free of other contaminants, which can include microbial contaminants. Further, as used herein, “purified bacterial strain” is intended to mean the strain separated from materials with which it is normally found in nature.

A “plant element” or “plant part” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, ear, spike, spikelet, fruit, stolon, bulb, tuber, corm, keikis, bud. As used herein, a “plant element” is synonymous to a “portion” of a plant, and refers to any part of the plant, and can include distinct tissues and/or organs, and may be used interchangeably with the term “tissue” throughout. In addition, a “plant element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

“Agricultural plants” or “plants of agronomic importance” include plants that are cultivated by humans for food, feed, fiber, fuel, and/or industrial purposes. In some embodiments, plants (including seeds and other plant elements) treated in accordance with the present invention are monocots. In a particular embodiment, the agricultural plant is selected from the group consisting of wheat (Triticum aestivum and related varieties), barley (Hordeum vulgare and related varieties) or maize (Zea mays and related varieties).

An “active formulation” refers to a mixture of chemicals that facilitate the stability, storage, and/or application of the bacterial strain(s). Treatment formulations may comprise any one or more agents such as: a carrier, a solvent, an adjuvant, an oil, an emulsifier, a spreader, a cryoprotectant, a binder, a dispersant, a surfactant, a buffer, a tackifier, a microbial stabilizer, a fungicide, a complexing agent, an herbicide, a nematicide, an insecticide, a plant growth regulator, a rodenticide, a desiccant, a nutrient, an excipient, a wetting agent, or a salt.

As used herein an “agriculturally compatible carrier” or “agriculturally compatible excipient” refers to any material, other than water, that can be added to a plant element without causing or having an adverse effect on the plant element (e.g., reducing seed germination) or the plant that grows from the plant element, or the like.

As used herein, a “colony-forming unit” or “CFU” is used as a measure of viable microorganisms in a sample. A CFU is an individual viable cell capable of forming on a solid medium a visible colony whose individual cells are derived by cell division from one parental cell.

The term “supernatant” refers to the liquid broth remaining when cells grown in said broth are removed by centrifugation, filtration, sedimentation or other means well known in the art.

The term “extract” refers to various forms of microbial products. Said microbial products are obtained by removing the cell walls and/or cell membranes of the bacterial strains, a process known as lysis, thereby obtaining one or more endogenous products of the bacterial strains in culture.

The term “treatment” as used herein refers to the reduction or elimination of the severity of a symptom of the disease, the frequency with which such a symptom is exhibited, or both.

The terms “prophylaxis” or “prophylactic” as used herein refer to completely or partially preventing or inhibiting a symptom of the disease or the frequency with which such a symptom is exhibited.

### DESCRIPTION OF FIGURES

The following description of the figures of specific embodiments of the invention is merely exemplary in nature and is not intended to limit the present teachings, their application, or uses.

FIGS. 1 to 3 show dose-response curves observed when testing the C₁₇H₂₇NO₄S compound according to Formula I or Formula II of the present invention against spores of fungal plant pathogens Fusarium graminearum (FIG. 1), Puccinia strilformis (FIG. 2), and Zymoseptoria tritici (FIG. 3), either as the activity as measured by absorbance (600 nm; cell viability/hyphal growth—FIGS. 1A and 3A) or as the activity as measured by fluorescence of a resazurin dye (excitation at 570 nm, emission at 615 nm; metabolic activity—FIGS. 1B, 2 and 3B).

### Deposit Information

The bacterial strains of current invention are deposited on 21 Aug. 2019 (B/00234,B/00233, and B/00235) and 8 Jun. 2020 (B/00309) with the Polish Collection of Microorganisms (Institute of Immunology and Experimental Therapy, Polish Academy of Sciences, Ul. Weigla 12, 53-114 Wroclaw, Poland), under the terms of the Budapest Treaty with Deposit ID: B/00234, B/00233, B/00235, and B/00309.

The biological material shall be made available as provided for under Rule 13bis.6 PCT and Rule 32(1) EPC only by the issuance of a sample to an Expert.

### DETAILED DESCRIPTION OF THE INVENTION

The present invention concerns a novel compound and its use.

In a first aspect, the invention relates to a compound wherein said compound is a compound according to Formula I or Formula II, or an acceptable salt thereof:

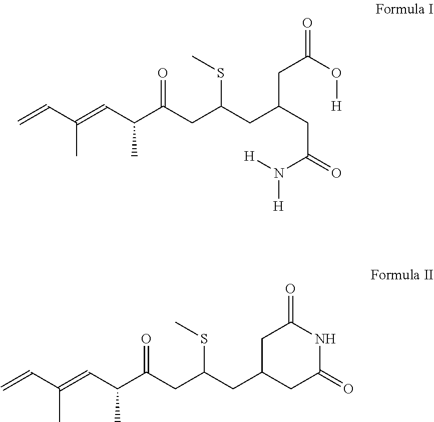 [SMILES: C=C/C(C)=C/[C@@H](C)C(=O)CC(CC(CC(N)=O)CC(=O)O)SC ; C=C/C(C)=C/[C@@H](C)C(=O)CC(CC1CC(=O)NC(=O)C1)SC]

The compound can either be composed of a polyketide structure and a closed glutarimide ring as shown in Formula II or being composed of a polyketide structure and the open form of a glutarimide ring (Formula I).

The compound of the present invention may comprise any acceptable salt of a molecule according to Formula I, including, but not limited to aluminum, calcium, magnesium, potassium, sodium, and the like.

In an embodiment, the compound according to Formula I or Formula II is produced or biosynthesized by a bacterial strain, preferably a strain of Streptomyces sp., such as selected from the list comprising Streptomyces hygroscopicus, Streptomyces antibioticus, Streptomyces coelicolor, Streptomyces peucetius, Streptomyces aureofaciens, Streptomyces ederensis, Streptomyces amphibiosporus, Streptomyces platensis, Streptomyces griseus or Streptomyces monomycini. In a preferred embodiment, said compound is produced by Streptomyces monomycini.

The inventors unexpectedly found that Streptomyces sp. were able to synthesize a hitherto unknown compound as metabolite with attractive features such as fungicidal activities. Hence, a method of obtaining said compound according to Formula I or Formula II is by purifying the metabolite from those strains that produce the latter. Accordingly in an embodiment, the current invention also relates to strains that produce the compound according to Formula I and/or Formula II.

Examples or representatives of such strains have been deposited under the Budapest Treaty with the Polish Collection of Microorganisms, as Deposit ID: B/00234,B/00233, B/00235 or B/00309. In an embodiment, the invention thus also relates to a mutant of a strain deposited as B/00234, B/00233, B/00235 or B/00309, said mutant is still capable of producing said compound; or to a strain having at least 98%, more preferably at least 98.1%, more preferably at least 98.2%, more preferably at least 98.3%, more preferably at least 98.4%, more preferably at least 98.5%, more preferably at least 98.6%, more preferably at least 98.7%, more preferably at least 98.8%, more preferably at least 98.9%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, more preferably at least 99.9% genomic sequence identity with a bacterial strain as deposited as Deposit ID: B/00234, B/00233,B/00235 or B/00309 and still capable of producing said compound.

In another embodiment, the compound having Formula I or Formula II or an acceptable salt thereof is artificially synthesised by means known in the art.

The synthesis of the compound can be done using the methods and means known in the art such as chemical synthesis. Chemical synthesis can be defined by these processes by which one or more chemical reactions are performed with the aim of converting a reactant or starting material into a product or multiple products.

In a possible non-limitative embodiment, said compound can be formed as an aldol coupling product of a ketone and the known aldehyde, the glutarimide group.

In an embodiment, the compound having Formula I or Formula II, an acceptable salt or a mixture thereof according to the previous embodiments is stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the compound is substantially stable at temperatures between about 4° C. and about 37° C. for at least about 5, 10, 15, 20, 25, 30 or greater than 30 days.

In another aspect, the present invention provides a composition comprising the compound with Formula I or Formula II, an acceptable salt or a mixture thereof of the invention. This composition is particularly suitable for crop protection and/or pharmaceutical and veterinary applications. Said compound can be according to any of the embodiments described above.

In an embodiment, said composition comprises said compound in a concentration of between 0.1% to 99.9%, between 0.1% and 90%, between 0.1% and 85%, between 0.1% and 80%, between 0.1% and 75, between 0.1% and 70%, between 0.1% and 65%, between 0.1% and 60%, between 0.1% and 55%, between 0.1% and 50%, between 0.1% and 45%, between 0.1% and 40%, between 0.1% and 35%, between 0.1% and 30%, between 0.1% and 25%, between 0.1% and 20%, between 0.1% and 15%, between 0.1% and 10%, between 0.1% and 5% or between 0.1% and 1%. In a further embodiment, said composition comprises said compound in a concentration of between 99% and 99.9%, between 95% and 99%, between 90% and 99%, between 85% and 99%, between 80% and 99.9%, between 75% and 99%, between 70% and 99%, between 65% and 99%, between 60% and 99%, between 55% and 99%, between 50% and 99%, between 45% and 99%, between 40% and 99%, between 35% and 99%, between 30% and 99%, between 25% and 99%, between 20% and 99%, between 15% and 99%, between 10% and 99%, between 5% and 99% or between 1% and 99%. In a further embodiment, said composition comprises said compound in a concentration of between 0.1% and 1%, between 0.1% and 2%, between 0.1% and 3%, between 0.1% and 4%, between 0.1% and 5%, between 0.1% and 6%, between 0.1% and 7%, between 0.1% and 8%, between 0.1% and 9% or between 0.1% and 10%. In a further embodiment, said composition comprises said compound in a concentration of between 1% and 2%, between 2% and 3%, between 3% and 4%, between 4% and 5%, between 5% and 6%, between 6% and 7%, between 7% and 8%, between 8% and 9%, between 9% and 10%, between 10% and 20%, between 20% and 30%, between 30% and 40%, between 40% and 50%, between 50% and 60%, between 60% and 70%, between 70% and 80%, or between 80% and 90%.

In an embodiment, the composition further comprises an excipient. Excipients are added to the formulation for a variety of purposes. By preference, said excipients—are chosen from fillers, binders, disintegrants, sweeteners, coatings, lubricants and/or glidants.

Diluents or fillers increase the bulk of a solid composition, and may make a dosage form containing the composition easier for the patient and care giver to handle.

Diluents suitable for tablets according to the current invention include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid compositions that are compacted into a dosage form, such as a tablet, may include excipients whose functions include helping to contain the active ingredient—and other excipients together after compression. Suitable binders include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®, pregelatinized starch, sodium alginate and starch.

The dissolution rate of a compacted solid composition may be increased by the addition of a disintegrant to the composition. Suitable disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, magnesium stearate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl furmarate, stearic acid, talc and zinc stearate. Preferably, said lubricant is present in between 0.25 and 1% w/w by weight.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid compositions may also be dyed using any pharmaceutically acceptable colorant to improve their appearance and/or facilitate, patient identification of the product and unit dosage level.

In an embodiment and specifically when said composition is intended for agricultural use, said excipient is an agricultural compatible excipient. Said “agricultural compatible carrier” or “agricultural compatible excipient” which can be regarded as a vehicle, is generally inert and it must be acceptable in agriculture. Thus, the phrase “agriculturally compatible” denotes a substance that can be used routinely under field conditions without interfering with growers' planting equipment, and without adversely influencing crop development or the desired ecological balance in a cultivated area.

The agriculturally compatible carrier or excipient can be solid. Solid carriers or excipients can include but are not limited to clays, natural or synthetic silicates, silica, resins, waxes, solid fertilizers, a polymer, a granular mass, perlite, a perlite granule, peat, a peat pellet, soil, vermiculite, charcoal, sugar factory carbonation press mud, rice husk, carboxymethyl cellulose, fine sand, calcium carbonate, flour, alum, a starch, talc, polyvinyl pyrrolidone, or a combination thereof. The agriculturally compatible carrier or excipient can be a liquid. Liquid carriers or excipients can include but are not limited to water, alcohols, ketones, petroleum fractions, oils, aromatic or paraffinic hydrocarbons, chlorinated hydrocarbons, liquefied gases or a combination thereof. More particularly, the agriculturally compatible carrier or excipient can include a dispersant, a surfactant, an additive, a thickener, an anti-caking agent, residue breakdown, a composting formulation, a granular application, diatomaceous earth, a coloring agent, a stabilizer, a preservative, a polymer, a coating or a combination thereof. One of the ordinary skills in the art can readily determine the appropriate carrier or excipient to be used taking into consideration factors such as a particular compound, plant to which the inoculum is to be applied, type of soil, climate conditions, whether the inoculum is in liquid, solid or powder form, and the like. The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof. The proteinaceous material can include a milk product, wheat flour, soybean meal, blood, albumin, gelatin, or a combination thereof. The thickener can comprise a long chain alkylsulfonate of polyethylene glycol, polyoxyethylene oleate or a combination thereof. The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcolhol, an alkyl phosphate, or a combination thereof. The anti-caking agent can include a sodium salt such as a sodium sulfite, a sodium sulfate, a sodium salt of monomethyl naphthalene sulfonate, or a combination thereof; or a calcium salt such as calcium carbonate, diatomaceous earth, or a combination thereof.

In an embodiment, said composition further comprises one or more of the following: water, other nutritive substance, weak acid, vegetable oil, essential oil, metabolism stimulant, emulsifying agent, viscosity agent, tinting material, suspending agent, dispersion agent, preservative, complexing agent, stabilizer, carrier, vehicle or wetting agent, or any combination thereof. In an embodiment, said composition further comprises at least one oil, surfactant and polymer.

In another embodiment, said composition, further comprises active ingredients such as fungicides, antibiotics, nematicides, herbicides and fertilizing agents. Preferably, said composition further comprises one or more of the following: fungicide, nematicide, bactericide, insecticide, molluscicide, algicide, herbicide, fertilizer, micronutrient fertilizer material, plant growth stimulator, stabilizer, preservative, carrier or excipient, complexing agent, bacterial inoculum, a plant growth amendment or any combination thereof. Non-limiting examples are provided above. In some cases said fertilizer is a liquid fertilizer. Liquid fertilizer can include without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, hampene (chelated iron), dolomitic limestone, hydrate lime, calcium carbonate, diammonium phosphate, monoammonium phosphate, potassium nitrate, potassium bicarbonate, monopotassium phosphate, magnesium nitrate, magnesium sulfate, potassium sulfate, potassium chloride, sodium nitrates, magnesian limestone, magnesia, disodium dihydromolybdate, cobalt chlorid hexahydrate, nickel chloride hexahydrate, indole butyric acid, L-tryptophan, urea, urea-formaldehydes, urea ammonium nitrate, sulfur-coated urea, polymer-coated urea, isobutylidene diurea, K2SO4-2MgSO4, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion or a combination thereof. The micronutrient fertilizer material can comprise boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate or a combination thereof. The insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof. The herbicide can comprise a chlorophenoxy compound, a nitrophenolic compound, a nitrocresolic compound, a dipyridyl compound, an acetamide, an aliphatic acide, an anilide, a benzamide, a benzoic acid, a benzoic acid derivative, anisic acid, an anisic acid derivative, a benzonitrile, benzothiadiazinone dioxide, a thiocarbamate, a carmabate, carbanilate, chloropyridinyl, a cyclohexenone derivative, a dinitroaminobenzene derivative, a fluorodinitrotoluidine compound, isoxazolidinone, nicotinic acide, isopropylamine, an isopropulamine derivative, oxadiazolinone, a phosphate, a phthalate, a picolinic acid compound, a triazine, a triazole, a uracil, a urea derivative, endothall, sodium chlorate, or a combination thereof. The fungicide can comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof.

Said composition may include microorganisms, such as abacterial inoculant or fungal inoculant, preferably which are shown to elicit a beneficiary action to a plant, e.g. pest or pathogen control. More preferably said inoculants also show a positive impact on plant growth and/or yield.

Without wishing to be limitative, said composition may comprise a fungal inoculant of the family Glomeraceae, a fungal inoculant of the family Claroidoglomeraceae, a fungal inoculant of the family Acaulosporaceae, a fungal inoculant of the family Sacculospraceae, a fungal inoculant of the family Entrophosporaceae, a fungal inoculant of the family Pacidsproraceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporacea, a fungal inoculant of the family Scutellosproaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, a fungal inoculant of the genus Glomus or a combination thereof.

Without wishing to be limitative, said composition may comprise a bacterial inoculant of genus Rhizobium, bacterial inoculant of the genus Bradyrhizobium, bacterial inoculant of the genus Mesorhizobium, bacterial inoculant of the genus Azorhizobium, bacterial inoculant of the genus Allorhizobium, bacterial inoculant of the genus Burkholderia, bacterial inoculant of the genus Sinorhizobium, bacterial inoculant of the genus Kluyvera, bacterial inoculant of the genus Azotobacter, bacterial inoculant of the genus Pseudomonas, bacterial inoculant of the genus Azosprillium, bacterial inoculant of the genus Bacillus, bacterial inoculant of the genus Streptomyces, bacterial inoculant of the genus Paenibacillus, bacterial inoculant of the genus Paracoccus, bacterial inoculant of the genus Enterobacter, bacterial inoculant of the genus Alcaligenes, bacterial inoculant of the genus Mycobacterium, bacterial inoculant of the genus Trichoderma, bacterial inoculant of the genus Gliocladium, bacterial inoculant of the genus Klebsiella, or a combination thereof.

In an embodiment, said composition is in a liquid, semisolid, solid or gaseous form. In another or further embodiment, said composition is in a dosage form of a tablet, capsule, powder, granulate, aerosol, paste, syrup, suspension, emulsion or solution. Non-limiting examples of said composition are soluble powders, soluble granules, wettable granules, tablet formulations, dry flowables, aqueous flowables, wettable dispersible granules, oil dispersions, suspension concentrates, dispersible concentrates, emulsifiable concentrates, aqueous suspensions, a fertilizer granule, or a sprayable composition. Said composition may be formulated such that it is suited for oral, injectable, intravenous, intramuscular, cutaneous, inhalation, topical or intranasal administration. In other embodiment, said composition is formulated to allow coating of one or more objects, spraying, spray coating, evaporating, nebulising, atomising, suspending, diluting, etc.

In an embodiment, said composition is shelf-stable. Optionally, the shelf-stable composition is in a dry formulation, a powder formulation, or a lyophilized formulation. In some embodiments, the composition is formulated to provide stability for the compound. In one embodiment, the composition is substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the composition is substantially stable at temperatures between about 4° C. and about 37° C. for at least about 5, 10, 15, 20, 25, 30 or greater than 30 days.

In a third aspect, the present invention also relates to a microorganism, wherein said microorganism produces a compound having Formula I or Formula II, an acceptable salt or a mixture thereof.

In a preferred embodiment, the microorganism producing the compound with Formula I or Formula II belongs to the group of Streptomyces sp. Without being limitative, said compound producing microorganism is one or more of the fallowing: Streptomyces hygroscopicus, Streptomyces antibioticus, Streptomyces coelicolor, Streptomyces peucetius, Streptomyces aureofaciens, Streptomyces ederensis, Streptomyces amphibiosporus, Streptomyces platensis, Streptomyces monomycinior Streptomyces griseus.

In a further preferred embodiment the microorganism producing said compound is Streptomyces monomycini.

In a further preferred embodiment, said strain is a strain as deposited under the Budapest Treaty with the Polish Collection of Microorganisms, as Deposit ID: B/00234, B/00233, B/00235 or B/00309; or is a mutant of a strain deposited under B/00234, B/00233, B/00235 or B/00309, said mutant is still capable of producing said compound; or a strain having at least 98%, more preferably at least 98.1%, more preferably at least 98.2%, more preferably at least 98.3%, more preferably at least 98.4%, more preferably at least 98.5%, more preferably at least 98.6%, more preferably at least 98.7%, more preferably at least 98.8%, more preferably at least 98.9%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, more preferably at least 99.9% genomic sequence identity with a bacterial strain as deposited as Deposit ID: B/00234, B/00233, B/00235 or B/00309 and still capable of producing said compound.

In an embodiment, said bacterial strain is soil sourced and further propagated outside its original environment.

In another aspect, the present invention relates to the compound having Formula I or Formula II, an acceptable salt or a mixture thereof, the composition thereof or the microorganism producing the compound as described in previous embodiments, for therapeutic and/or prophylactic use. Said compound, composition or microorganism is particularly suitable for pharmaceutical and veterinary applications in humans and animals.

In particular, said compound composition comprising said compound or the microorganism producing said compound is used in the treatment and/or prophylaxis of bacterial and fungal infections in a subject or as an antimicrobial agent. In a particularly preferred embodiment, said compound, composition on microorganism is used in a suitable pharmaceutical dosage form to treat or prevent bacterial and/or fungal infections in a subject. To that purpose, and specifically when use in a human or animal subject is envisioned, said compound, composition or microorganism may be formulated into a composition for oral, injectable, intravenous, intramuscular, cutaneous, inhalation, topical or intranasal administration and/or formulated as a tablet, capsule, powder, suspension of liquid, emulsion, cream or aerosol.

In a specific embodiment, use in plant protection is envisioned. Said plant can be a monocotyledonous and dicotyledonous plant, including fodder or forage legumes, ornamental plants, food crops, trees or shrubs. In an embodiment, said plant is preferably a crop. Preferably, said plant belongs to the group of Acer spp., Actinidiaspp., Abelmoschus spp., Agave sisalana, Agropyron spp., Agrostis stolonifera, Alliumspp., Amaranthus spp., Ammophila arenaria, Ananas comosus, Annona spp., Apium graveolens, Arachis spp., Artocarpus spp., Asparagus officinalis, Avena spp., Averrhoa carambola, Bambusa sp., Benincasa hispida, Bertholletia excelsea, Beta vulgaris, Brassica spp., Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegusspp., Crocus sativus, Cucurbita spp., Cucumis spp., Cynara spp., Daucus carota, Desmodium spp., Dimocarpus longan, Dioscorea spp., Diospyros spp., Echinochloaspp., Elaeis sp., Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp., Gossypium hirsutum, Helianthus spp., Hemerocallis fulva, Hibiscus spp., Hordeum spp., Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp., Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp., Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp., Tropaeolum minus, Tropaeolum majus, Vaccinium spp., Vicia spp., Vigna spp., Viola odorata, Vitis spp., Zea mays, Zizania palustris, Ziziphus spp.; including the progenies and hybrids between the above. More preferably said plant is a crop, most preferably said plant is Triticum spp., Secale spp., Hordeum spp. or Avena spp.

Preferably said plant pathogen belongs to the group of Phytophthora infestans, Xanthomonas spp. (campestris), Pseudomonas syringae, Rhizobium spp., Tilletia tritici, Claviceps purpurea, Oculimacula spp., Fusarium spp., Phytium spp., Erysiphe graminis, Puccinia graminis, Puccinia triticina, Puccinia striiformis, Pyrenophora tritici-repentis, Ramularia spp. (collo-cygni), Sclerotinia sclerotiorum, Botrytis cinerea, Rhizoctonia solani, Colletotrichum graminicola, Microdochium nivale, Gaeumannomyces graminis var. tritici, Tapesia spp., Thysanoptera spp., Ustilago spp. or Mycosphaerella spp.

In an particularly advantageous embodiment, use in post-harvest treatment, wherein post-harvest is defined as the stage of plant or crop production immediately following harvest, including cooling, cleaning, sorting and packing. The instant a crop is removed from the ground, or separated from its parent plant, it begins to deteriorate. Postharvest treatment of a crop largely determines final quality, whether a crop is sold for fresh consumption, or used as an ingredient in a processed food product.

In an embodiment said compound, composition or microorganism as described before is used in integrated pest management programs.

“Integrated pest management” (IPM), also known as “integrated pest control” (IPC), is a broad-based approach that integrates practices for economic control of pests. IPM aims to suppress pest populations below the economic injury level (EIL). The UN's Food and Agriculture Organization defines IPM as “the careful consideration of all available pest control techniques and subsequent integration of appropriate measures that discourage the development of pest populations and keep pesticides and other interventions to levels that are economically justified and reduce or minimize risks to human health and the environment. IPM emphasizes the growth of a healthy crop with the least possible disruption to agro-ecosystems and encourages natural pest control mechanisms”.

Said compound, composition or microorganism can be applied to the soil, plant, seed, rhizosphere, or other areas of the plant.

Consequently, the present invention also relates to a method of controlling a pest or pathogen in plants which comprises introducing the compound, the composition comprising said compound or a microorganism producing said compound according to any of the above embodiments to a plant, a plant part or a substrate comprising or hosting said plant, thereby conferring pathogen resistance or pathogen control to said plant. Said introduction may be artificially.

Methods for introducing the compound, the composition thereof or the microorganism producing it to plants may include: treating the plant and/or a plant part and/or growth medium wherein said plant is grown, inoculation of thee seeds, coating the seeds, direct inoculation of plants or plant parts, spraying or wetting plants or plant parts (eg. ears). Preferably, a wheat ear, spike, spikelet, stem and/or leaf is treated. An appropriate method may be chosen depending on the type of plant to which the compound, composition or microorganism is to be introduced.

As another of further non-limiting example, the compound, composition thereof or microorganism producing it may be applied in the form of coatings. The coating may be applied to the seed by spraying on the seeds or soaking said seeds in a solution containing the compound, composition thereof or microorganism producing it. In another example, for coating seeds with a solution as described in the previous example, a binding agent may be added, such as a binding agent comprising carbide (calcium carbonate).

In embodiments, the coating may be applied to a naked and untreated plant part. In other embodiments, the coating may be applied as an overcoat to a previously treated plant part. Seed coatings are particularly preferred in the treatment of soil borne fungal diseases. In embodiments, the seed coating may be applied to a naked and untreated seed. In other embodiments, the seed coating may be applied as a seed overcoat to a previously treated seed.

In an embodiment, the compound, composition thereof or microorganism may be applied to the soil or any other substrate in which said plant grows in order to remove pests and/or pathogen from said substrate.

Inoculating the substrate comprising or hosting said plant or plant part can be performed, by way of example and without the intention to be limiting, using a powder, a granule, a pellet, a plug, or a soil drench that is applied to the substrate. Inoculation could also be performed by a liquid application, such as a foliar spray or liquid composition. The application may be applied to a growing plant or to the substrate. Plants, in particular agricultural plants, can be grown in substrate. In one embodiment, said substrate is soil, sand, gravel, polysaccharide, mulch, compost, peat moss, straw, logs, clay, or a combination thereof. In another embodiment, the substrate can also include a hydroculture system or an in vitro culture system. In some embodiments, a combination of different application methods as described herein is applied. In a non-limiting example, wheat plants are treated against Fusarium spp. and Puccinia spp. by foliar application. Also strawberry plants and tomato plants may for example be treated against Botrytis spp. by foliar application. In another non-limiting example, lettuce plants are treated against Rhizoctonia spp. and Pythium spp. by foliar application, subsequently followed by soil drench applications.

Applying said compound, composition thereof or microorganism, to a seed, the plant itself or its substrate modulates a trait of agronomic importance. The trait of agronomic importance can be amongst others pathogen or pest resistance, plant growth, and/or plant yield.

The invention is further described by the following non-limiting examples which further illustrate the invention, and are not intended to, nor should they be interpreted to, limit the scope of the invention.

### EXAMPLES

### Example 1: Structural Identification of the Compound by LC/HRMS

### 1. Culturing of Bacterial Strains

Purified bacterial strains deposited as B/00234, B/00233, B/00235 and B/00309were inoculated in four 500 ml Erlenmeyer containers, in 125 ml bacteria growth medium and were incubated for 2-5 days at 28° C. and 70% humidity on an orbital shaker (220 rpm).

### 2. HPLC Fractionation

The mycelium and fermentation broth in flasks were extracted by adding acetone. After filtration, the acetone extract was concentrated under reduced pressure. The aqueous residue was loaded onto a resin column for a solid-phase extraction (SPE) and eluted with acetone. The extract was evaporated and the residue solved in 100% DMSO and filtered at 0.2 mM prior to semi-preparative fractionation to constitute a HPLC head. An aliquot of the flow-though was kept for its possible evaluation of activity if not retained by the SPE resin.

100 mg per extract were then subjected to semi-preparative reversed phase HPLC (Zorbax SB-C8 column, 21.2×250 mm, 7 um, 20 mL/min, UV detection at 210 and 280 nm) eluting with CH₃CN: H₂O, in a linear gradient of acetonitrile specific for each sample, and a wash step to yield a collection of 80 fractions. After evaporation of the organic solvent, fraction aliquots of 100 mg/ml in 20% of DMSO in water were prepared.

### 3. LC/HRMS De-replication of the Compound

The fractions were subsequently analyzed via Liquid Chromatography-High Resolution Mass Spectrometry (LC-HRMS) using an HPLC machine (detection at 210 nm) interfaced to a mass spectrometer. A Zorbax was used for the separation. Two solvents were used as mobile phase: solvent A water:AcN 90:10, solvent B water:AcN 10:90, both with 13 mM ammonium formate and 0.01% TFA.

The retention time and exact mass of the detected components were compared against high-resolution mass spectrometry databases. Based thereon, the compounds have been identified. Among other known compounds, the compound of the present invention was identified and the molecular formula resolved: C₁₇H₂₇NO₄S. This corresponds to a compound with Formula I or II as described herein.

### Example 2: Screening the Compound Against the Wheat Pathogens Zymoseptoria tritici, Fusarium graminearum and Puccinia striiformis

To assess the antifungal activity of said compound, three fungal phytopathogenic strains were used, of which two were acquired from two public collections: CBS-KNAW culture collection, Utrecht (Netherlands) and CIRM-CF, French National Institute for Agricultural Research, Marseille (France).

1. Fusarium graminearum Agar-based Screening Assay

The medium used for agar antifungal evaluation was SDA (sabouroad dextrose agar Difco™ 65 g and 1000 mL H2O miliQ). Fusarium spores inoculated media was dispensed in assay plates. Subsequently, a compound with Formula I or II was dispensed on the agar surface. Plates were incubated for growth and inhibition halos were measured by the diameter and opacity to assess antimicrobial activity.

Results: Inhibitory activity was observed on the colonies of Fusarium graminearum.

2. Zymoseptoria tritici and Puccinia striiformis Liquid-based Screening Assays

Liquid-based assays were performed in 96-well plates. Each well contained 10 μL of extract fraction and 90 μL of specific medium containing conidia of Z. tritici or water agar containing 10 mg/ml of P. striiformis spores.

After specific incubation time for each fungal strain, the compound's antifungal activity was quantified by measuring the absorbance differences at 600 nm between the final and the initial incubation times after the addition of resazurin, an oxidation-reduction indicator of the cell viability. All assays were performed per duplicate. The color of the wells after resazurin addition was also recorded using a Canon camera. A blue color is interpreted as absence of metabolic activity (low ratio of conidia germination). A fluorescent pink color is interpreted as presence of metabolic activity (high ratio of conidia germination). A purple color in the well is interpreted as a trailing result, where some metabolic activity was present, but a longer incubation time may allow the purple color to change to pink. An extract or fraction is considered as active when its percentage of inhibition are superior to 50%.

Results: Inhibitory activity was observed on P. striiformis and Z. tritici.

### Example 3: Activity of the Purified Compound Against Three Plant Pathogens

Although the below example refers to the purified bacterial strain as deposited as B/00234, the experiments have been repeated using the purified bacterial strains B/00233, B/00235 and B/00309, and very similar results have been obtained.

### 1. Culturing of Bacterial Strains

The purified bacterial strain deposited as B/00234 was seeded in eighty 500 ml Erlenmeyer containers, in 125 ml bacteria growth medium and was incubated for 7 days at 28° C. at 70% relative humidity on an orbital shaker (220 rpm).

### 2. HPLC Fractionation and Purification

The mycelium and fermentation broth in flasks were extracted by adding acetone. After filtration, the acetone extract was evaporated under a nitrogen stream. The aqueous residue was loaded onto a resin column for a solid-phase extraction (SPE) and eluted with acetone. After HRMS analyses, the fractions containing the C₁₇H₂₇NO₄S compound were pooled in three pools and lyophilized. 150 mg of each pool were chromatographed in a HPLC preparative step. HPLC fractions were then analyzed by HRMS to determine the relative presence of the C₁₇H₂₇NO₄S compound.

Repeated preparative HPLC was performed on the two pools containing the highest amount of the compound. HPLC sub-fractions containing the target molecule were combined and repurified using the same methodology. Fractions were purified in semipreparative HPLC until they reached a purity >90%. Identity of the compound was then checked by HRMS and after confirmation, all fractions but one were titrated in DMSO; the last fraction being used for NMR identity confirmation of the C₁₇H₂₇NO₄S compound.

3. Inhibition Halo Diameter Obtained Against F. graminearum in an Agar-based Assay

Spores of F. graminearum were mixed with 25 ml of a still-liquid SDA growing medium, then poured in assay plates. When the growing medium had solidified, compounds used in the test were pipetted onto the medium. The compound C₁₇H₂₇NO₄S was titrated in 100% DMSO at 8 mg/mL in two-fold dilutions (10 points) and 2 μL of each were dispensed in assay plates and incubated in dark at 25° C. and 70% RH for 24 hours. Amphotericin B was used as a positive control. The diameter and opacity of inhibition halos were measured to assess antimicrobial activity with an image analysis software (Image Analyzer®) and a magnifying glass (Leica™ MZ16). The opacity was scored using the software as follows: ‘A’ means 80-100% inhibition of the pathogen, ‘B’ means 60-80% inhibition of the pathogen, ‘C’ means 40-60% inhibition of the pathogen, ‘D’ means 20-40% inhibition of the pathogen, and ‘E’ means 0-20% inhibition of the pathogen.

The compound C₁₇H₂₇NO₄S was shown to be active against F. graminearum spores at all concentrations tested (8 mg/mL to 125 μg/mL), as shown in the Table 1 below.

TABLE 1

| | Volume | | Halo | |
|---|---|---|---|---|
| | pipetted onto | Concentration | Diameter | Opacity |
| Sample | the plate (μL) | (mg/mL) | (mm) | Grade |
| Amphotericin B | 10 | 0.250 | 9.2 | A |
| Amphotericin B | 10 | 0.250 | 9 | A |
| Amphotericin B | 10 | 0.250 | 9.3 | A |
| Amphotericin B | 10 | 0.250 | 8.7 | A |
| Amphotericin B | 10 | 0.125 | 9.3 | B |
| Amphotericin B | 10 | 0.625 | 9.1 | B |
| Amphotericin B | 10 | 0.3125 | 7.7 | C |
| Amphotericin B | 10 | 0.15625 | 7.1 | C |
| C₁₇H₂₇NO₄S | 2 | 8 | 9.8 | A |
| C₁₇H₂₇NO₄S | 2 | 8 | 9.2 | A |
| C₁₇H₂₇NO₄S | 2 | 8 | 9.7 | A |
| C₁₇H₂₇NO₄S | 2 | 4 | 9.5 | A |
| C₁₇H₂₇NO₄S | 2 | 4 | 9.2 | A |
| C₁₇H₂₇NO₄S | 2 | 4 | 9.6 | A |
| C₁₇H₂₇NO₄S | 2 | 2 | 9.1 | A |
| C₁₇H₂₇NO₄S | 2 | 2 | 9.3 | A |
| C₁₇H₂₇NO₄S | 2 | 2 | 9.1 | A |
| C₁₇H₂₇NO₄S | 2 | 1 | 8.3 | A |
| C₁₇H₂₇NO₄S | 2 | 1 | 8.3 | A |
| C₁₇H₂₇NO₄S | 2 | 1 | 8.2 | A |
| C₁₇H₂₇NO₄S | 2 | 0.500 | 6.7 | B |
| C₁₇H₂₇NO₄S | 2 | 0.500 | 7.2 | B |
| C₁₇H₂₇NO₄S | 2 | 0.500 | 7.1 | B |
| C₁₇H₂₇NO₄S | 2 | 0.250 | 5.5 | D |
| C₁₇H₂₇NO₄S | 2 | 0.250 | 5.6 | D |
| C₁₇H₂₇NO₄S | 2 | 0.250 | 5.5 | D |
| C₁₇H₂₇NO₄S | 2 | 0.125 | 4.4 | E |
| C₁₇H₂₇NO₄S | 2 | 0.125 | 4.7 | E |
| C₁₇H₂₇NO₄S | 2 | 0.125 | 4.3 | E |

4. Dose-response Antifungal Activity of the C₁₇H₂₇NO₄S Compound Against Fusarium graminearum, Puccinia striiformis and Zymoseptoria tritici in Liquid-based Assays

The compound C₁₇H₂₇NO₄S was tested in liquid-based assays against spores of Fusarium graminearum, Puccinia striiformis and Zymoseptoria tritici. 96-well plates were loaded as follows: each well contained 2 μL of the compound at different concentrations and 98 μL of the target fungal pathogen. The compound was tested in two-fold dilutions (10 points) dissolved in 100% DMSO and 2 μL of each point was dispensed in the assay plate per duplicate. Positive controls (amphotericin B for F. graminearum and Z. tritici; eugenol for P. striiformis) and negative controls (DMSO 2%) were included in each plate.

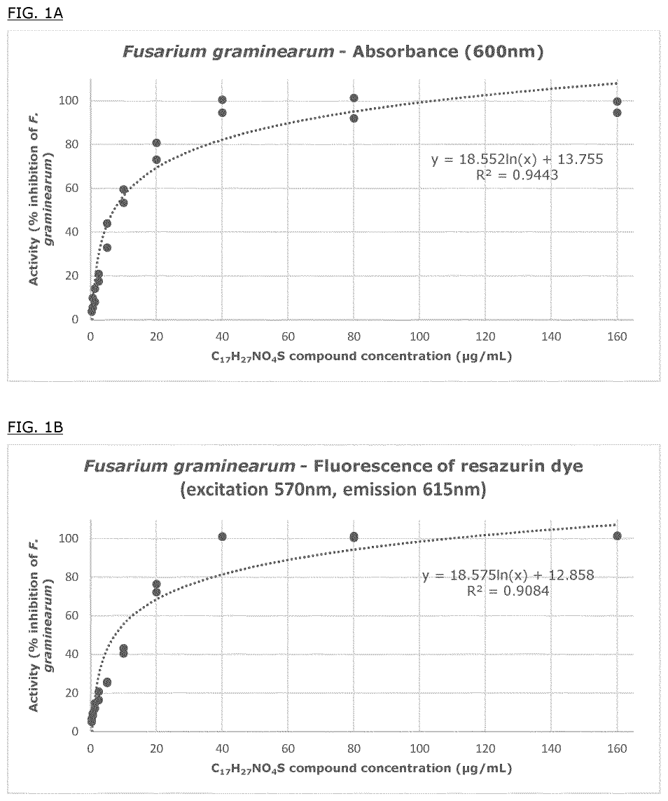

After a specific incubation time for each fungal strain, the compound's antifungal activity was quantified by measuring the absorbance differences at 600 nm between the final and the initial incubation times after the addition of resazurin, an oxidation-reduction indicator of the cell viability. Results of the dose-response curve are shown in FIGS. 1A (F. graminearum) and 3A (Z. tritici), and below in Table 2. Based on this curve, an estimated AC50 value (the dose/concentration that induces half-maximum response—in the present case inhibition of the pathogen) and the measured range of the AC50 values is given in the table below. An extract or fraction is considered as active when its percentage of inhibition is superior to 50%. The absorbance (at 600 nm) provided a measure of the hyphal growth within the wells. For P. striiformis, the absorbance was not measured (indicated as ‘/’) as the pathogen is an obligate biotroph and produces too few germinative hyphae under in vitro conditions for absorbance to be a relevant metric.
the color of the wells after resazurin addition, an indication of metabolic activity, was recorded using a Canon camera. A blue color is interpreted as the absence of metabolic activity. A fluorescent pink color is interpreted as the presence of metabolic activity. A purple color in the well is interpreted as a trailing result, where some metabolic activity was present, but a longer incubation time may allow the purple color to change to pink. The fluorescence was measured (excitation at 570 nm and emission at 615 nm) and the AC50 values were determined. An extract or fraction is considered as active when its percentage of inhibition is superior to 50%. Contrary to measuring absorbance at 600 nm, measuring the fluorescence of the dye is relevant for P. striiformis too, and was therefore assessed. Results are given in FIGS. 1B (F. graminearum), 2 (P. striiformis) and 3B (Z. tritici), and Table 2 below.

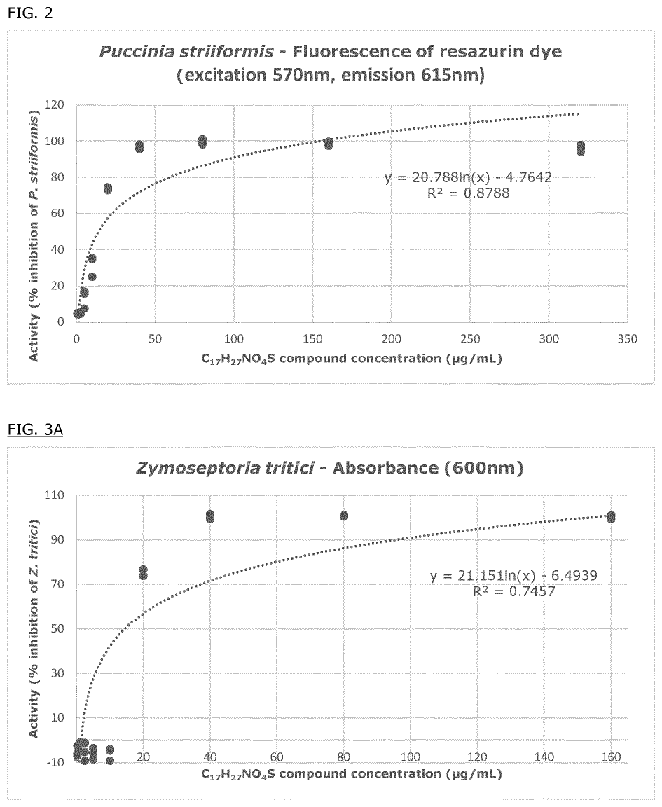

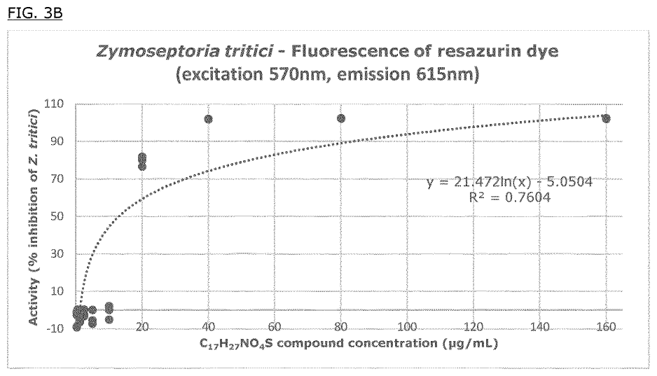

The results show that the C₁₇H₂₇NO₄S compound showed activity against all three pathogens (Table 2 and FIGS. 1 to 3) at relatively low concentrations ranging from 7.54 to 17.21 μg/mL for hyphal growth in the wells, and ranging from 11.66 to 17.08 μg/mL for metabolic activity.

TABLE 2

| | | | | | Fluorescence of | |
|---|---|---|---|---|---|---|
| | | | | | resazurin dye | |
| | Dose- | | | | (excitation 570 nm, | |
| | response | Incubation | Absorbance (600 nm): | | emission 615 nm): | |
| | curve | time | AC50 value | | AC50 value | |
| Fungal | start | before | Estimation | Range | Estimation | Range |
| pathogen | (μg/mL) | readout | (μg/mL) | (μg/mL) | (μg/mL) | (μg/mL) |
| Fusarium | 160 | 22 h | 8.9 | 7.54-10.50 | 13.24 | 11.81-14.84 |
| graminearum | | | | | | |
| Puccinia | 320 | 2 d | / | / | 12.79 | 11.66-14.03 |
| striiformis | | | | | | |
| Zymoseptoria | 160 | 5 d | 16.02 | 14.92-17.21 | 15.90 | 14.80-17.08 |
| tritici | | | | | | |

1. (canceled)

2. (canceled)

3. (canceled)

4. A composition comprising a compound according to Formula I or Formula II, or an acceptable salt thereof: 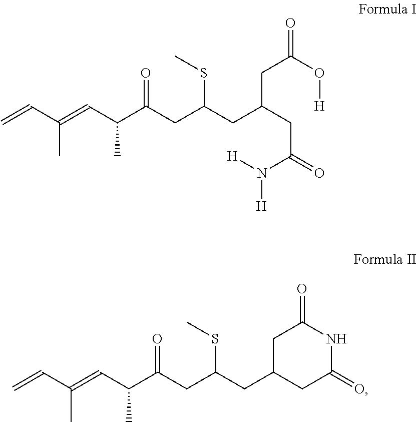 [SMILES: C=C/C(C)=C/[C@@H](C)C(=O)CC(CC(CC(N)=O)CC(=O)O)SC ; C=C/C(C)=C/[C@@H](C)C(=O)CC(CC1CC(=O)NC(=O)C1)SC]
in a concentration of between 0.1% to 99.9%.

5. The composition according to claim 4, wherein said composition further comprises an excipient.

6. The composition according to claim 4, wherein said composition further comprises one or more active ingredients selected from fungicides, antibiotics, nematicides, herbicides and/or fertilizing agents.

7. The composition according to claim 4, wherein said composition is in a liquid, semisolid, solid or gaseous form and/or wherein said composition is in a dosage form of a tablet, capsule, powder, granulate, aerosol, paste, syrup, suspension, emulsion or solution.

8. (canceled)

9. (canceled)

10. (canceled)

11. (canceled)

12. (canceled)

13. (canceled)

14. A method of controlling a pathogen in plants, the method comprising the step of introducing the compound according to Formula I or Formula II, or an acceptable salt thereof: 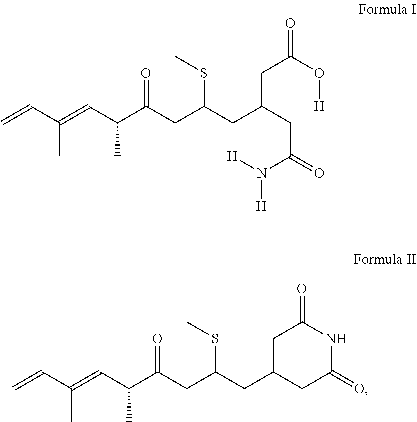 [SMILES: C=C/C(C)=C/[C@@H](C)C(=O)CC(CC(CC(N)=O)CC(=O)O)SC ; C=C/C(C)=C/[C@@H](C)C(=O)CC(CC1CC(=O)NC(=O)C1)SC]
to a plant, a plant part or a substrate comprising or hosting said plant, thereby conferring pathogen resistance or pathogen control to said plant.

15. The method of controlling a pathogen in plants according to claim 14, wherein said pathogen is selected from pathogens belonging to Phytophthora infestans; Xanthomonas sp. (campestris), Pseudomonas syringae, Rhizobium sp.; Tilletia tritici, Claviceps purpurea, Oculimacula spp., Fusarium sp., Phytium sp., Erysiphe graminis, Puccinia graminis, Puccinia triticina, Puccinia striiformis, Pyrenophora tritici-repentis, Ramularia sp. (collo-cygni), Sclerotinia sclerotiorum, Botrytis cinerea, Rhizoctonia solani, Colletotrichum graminicola, Microdochium nivale, Gaeumannomyces graminis var. tritici, Tapesia sp., Thysanoptera, Ustilago spp. or Mycosphaerella spp.

16. A method of treating a fungal infection in or on a subject, the method comprising the step of administering an effective amount of the compound according to Formula I or Formula II, or an acceptable salt thereof: 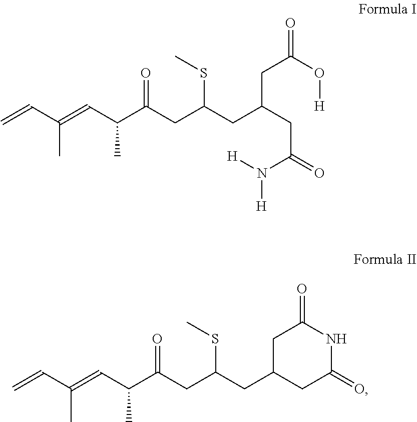 [SMILES: C=C/C(C)=C/[C@@H](C)C(=O)CC(CC(CC(N)=O)CC(=O)O)SC ; C=C/C(C)=C/[C@@H](C)C(=O)CC(CC1CC(=O)NC(=O)C1)SC]
to the subject.

17. A plant seed coated with a composition according to claim 4.

18. The composition according to claim 4, wherein the composition is an antifungal composition.

19. The composition according to claim 4, further comprising a Streptomyces monomycini strain producing the compound according to Formula I or II.

20. The composition according to claim 18, wherein said S. monomycini strain is selected from the strains deposited as Deposit ID: B/00234, B/00233, B/00235 or B/00309.

21. The composition according to claim 4, wherein said composition further comprises an agricultural compatible excipient.
